(19) United States
(12) Patent Application Publication
(10) Pub. No.: US 2010/0075432 A1
(43) Pub. Date: Mar. 25, 2010

(54) SENSOR

(76) Inventors: Sergey Anatoliyovich Piletsky, Bedforshire (GB); Olivier Yves Frederic Henry, Concarneau (FR); Khalku Karim, Cambridgeshire (GB); Peter Georg Laitenberger, Cambridgeshire (GB)

Correspondence Address: STITES & HARBISON PLLC, 1199 NORTH FAIRFAX STREET, SUITE 900, ALEXANDRIA, VA, 22314, US

(21) Appl. No.: 12/529,656

(22) PCT Filed: Feb. 29, 2008
(86) PCT No.: PCT/GB08/00700

§ 371 Date: Dec. 2, 2009

(30) Foreign Application Priority Data
Mar. 3, 2007 (GB) 0704150.2

(51) Int. Cl. G01N 33/53; B05D 3/00; G01N 27/00

(52) U.S. Cl. 436/131; 427/58; 422/82.01

(57) ABSTRACT

This invention relates to a sensor and in particular to a sensor for the detection of biologically important species. Specifically, the invention provides a sensor for detecting an analyte in a sample. The sensor comprises a transducer and a receptor layer in electrical communication with the transducer, wherein the receptor layer comprises a receptor material and a dispersed electrically conductive material

### TECHNICAL FIELD

This invention relates to a sensor and in particular to a sensor for the detection of biologically important species.

### BACKGROUND ART

Modern healthcare relies extensively on a range of chemical and biochemical analytical tests on a variety of body fluids to enable diagnosis and management of disease. Medical and technological advances have considerably expanded the scope of diagnostic testing over the past few decades. Moreover, an increasing understanding of the human body, together with the emergence of developing technologies, such as microsystems and nanotechnology, are expected to have a profound impact on diagnostic technology.

Increasingly, diagnostic tests in hospitals are carried out at the point-of-care (PoC), in particular, in situations, where a rapid response is a prime consideration and therapeutic decisions have to be made quickly. Despite recent advances in PoC testing, several compelling needs remain unmet. Many of the presently available diagnostic tests rely on the use of sophisticated biological receptors, such as enzymes, antibodies and DNA. Due to their biological derivation, these biomolecules typically suffer from a number of limitations when used in sensing applications, for example, poor reproducibility, instability during manufacture, sensitivity to environmental factors, such as pH, ionic strength, temperature etc., and problems associated with the sterilisation process.

A promising route to overcome these issues is offered by synthetic polymer-based receptors, such as molecularly imprinted polymers (MIPs). Synthetic receptors avoid many of the disadvantages associated with biological receptors. Molecular imprinting, for example, is a generic and cost-effective technique for preparing synthetic receptors, which combine high affinity and high specificity with robustness and low manufacturing costs. In addition, MIP receptor materials have already been demonstrated for a wide range of clinically relevant compounds and diagnostic markers. In contrast to biological receptors, synthetic receptors, and particularly MIPs, typically are stable at low and high pH, pressure and temperature, are inexpensive and easy to prepare, tolerate organic solvents, may be prepared for practically any analyte, and are compatible with micromachining and microfabrication technology.

Molecular imprinting may be defined as the process of template-induced formation of specific recognition sites (binding or catalytic) in a material, where the template directs the positioning and orientation of the material's structural components by a self-assembling mechanism. The material itself could be oligomeric, polymeric (for example, organic MIPs and inorganic imprinted silica gels) or two-dimensional surface assemblies (grafted monolayers).

In many applications, for example, where the receptor is to be used repeatedly without significant regeneration between applications, the use of so-called non-covalent MIPs is generally preferred, in particular in sensing applications. As the template/analyte is only weakly bound by non-covalent interactions to these receptor materials, it can be relatively easily removed from the synthetic receptor and the sensor regenerated for a new measurement. In general, non-covalent imprinting is easier to achieve and applicable to a wider spectrum of templates.

In non-covalent MIPs, the monomer(s) contained within the polymer interact with the template, i.e. the target analyte or a structural analogue thereof, through non-covalent interactions, for example, hydrogen bonding, electrostatic interaction, coordination-bond formation etc. FIG. 1 shows a schematic representation of the self-assembly of a MIP from monomeric starting materials to form a polymer having binding sites with specificity for the template and the subsequent elution or extraction of the template.

This technique has been employed to create successfully MIPs for a range of chemical compounds, ranging from small molecules (up to 1200 Da), such as small organic molecules (e.g. glucose) and drugs, to large proteins and cells. The resulting polymers are robust, inexpensive and, in many cases, possess affinity and specificity that is suitable for diagnostic applications. The high specificity and stability of MIPs render them promising alternatives to enzymes, antibodies, and natural receptors for use in sensor technology. See WO 2005/075995 for further details regarding MIPs and other synthetic polymers.

In WO 2005/075995, a sensor is described having a confinement structure, a receptor composed of a synthetic polymer, a substrate and a transducer. The confinement structure is disposed on the substrate and comprises a first limiting structure defining a first interior space. The transducer and the receptor are disposed in the first interior space. A second limiting structure defining a second interior space which encloses the first limiting structure may also be provided. The receptor is described as being proximal to the transducer and it is described that where the receptor is not in physical contact with the transducer, in the case of an amperometric transducer or a conductimetric transducer, electronic communication between the receptor and the transducer must be maintained, for example by the presence of a conducting polymer, electrically conducting organic salts or an electrolyte.

However, there remains a requirement in the art for still greater sensitivity in the sensor.

### DISCLOSURE OF INVENTION

Accordingly, the present invention provides a sensor for detecting an analyte in a sample comprising a transducer and a receptor layer in electrical communication with the transducer, wherein the receptor layer comprises a receptor material and a dispersed electrically conductive material.

That is, the sensor comprises a dispersed electrically conductive material which enhances electronic communication between the analyte and the receptor layer. This is in contrast to sensors of the type disclosed in WO 2005/075995 in which communication between the receptor and the transducer is considered, but not between the analyte and the receptor. The present invention therefore provides a continuous conduction path from the analyte to transducer via the receptor layer.

The present invention will now be described with reference to the accompanying drawings.

### BRIEF DESCRIPTION OF DRAWINGS

FIG. 1 shows a schematic representation of the self-assembly of a MIP and is discussed hereinabove with reference to the state of the art;

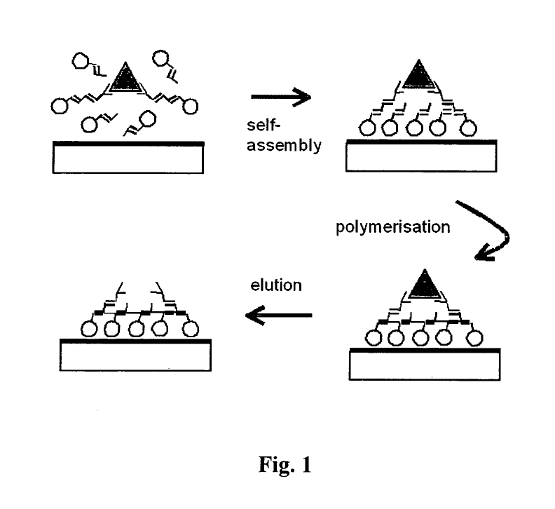

FIG. 2 shows a further schematic representation of a sensor in which FIG. 2A shows the sensor incorporating a MIP, FIG. 2B shows the same sensor as FIG. 2A with the addition of conductive material, and FIG. 2C shows a sensor having a bare transducer; and

FIG. 3 shows a sensor for performing the method of the present invention incorporated into an intravenous monitoring system.

### MODES FOR CARRYING OUT THE INVENTION

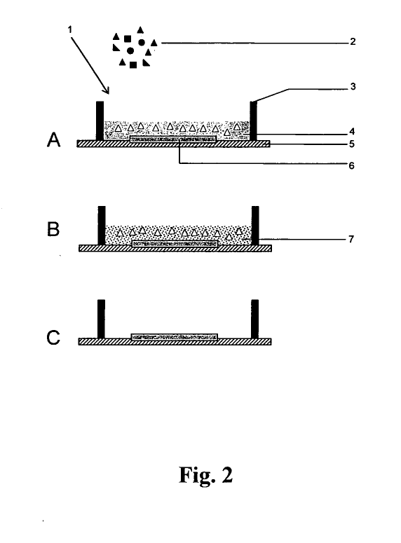

FIG. 2A shows a typical sensor 1 of the type used in the present invention for detecting the presence of an analyte in a sample 2. The sensor 1 comprises a confinement structure 3, a receptor layer 4, a substrate 5 and a transducer 6. Such a sensor is described in more detail in WO 2005/075995. The confinement structure 3 is disposed on the substrate 5. The confinement structure 3 comprises a first limiting structure defining a first interior space. As shown in FIG. 2A, the transducer 6 and the receptor layer 4 are disposed in the first interior space. The receptor layer is in communication with the transducer. Preferably the first limiting structure is a continuous structure, i.e. the walls are continuous and fully surround enclose the first interior space and most preferably is annular, i.e. a “well”. A second limiting structure defining a second interior space which encloses the first limiting structure may also be provided as described in WO 2005/075995. The first and second limiting structures are preferably composed of polyimide. The sensor 1 may further, comprise a channel to contain the sample and to define a flow path to direct the sample to the receptor layer 4.

In use, the sensor 1 is presented with the sample 2. The sample 2 is typically a fluid sample, preferably a liquid and most preferably a bodily fluid, such as blood. The sample is a “complex sample” in that it comprises the analyte being detected (represented in FIG. 2 by the equilateral triangles) as well as one or more interferents (represented by the squares, circles and right-angled triangles), which can interfere with the specific detection of the analyte.

Any material having a high binding affinity and selectivity for the analyte and which may be immobilised on a microsensor chip may be used as the receptor material in the receptor layer 4. Preferably the receptor layer 4 comprises a synthetic polymer, a biomolecule or a combination thereof, more preferably the receptor layer comprises an ionophore, a molecularly imprinted polymer (MIP), an enzyme, an antigen, an imprinted silica gel, a two-dimensional surface assembly (grafted monolayers) or a combination thereof, most preferably the receptor layer 4 comprises a MIP.

Suitable MIPs are described hereinabove and any of these MIPs may be incorporated in to receptor layer 4. By way of an example, where the analyte to be detected is propofol, the MIP is preferably a polymer based on one or more of the monomers N,N-diethylamino ethyl methacrylate (DEAEM), acrylamide, 2-(trifluoromethyl)acrylic acid (TFMAA), itaconic acid and ethylene glycol methacrylate phosphate (EGMP). The cross-linker is preferably selected from ethylene glycol dimethacrylate (EDMA), glycerol dimethacrylate (GDMA), trimethylacrylate (TRIM), divinylbenzene (DVB), methylenebisacrylamide and piperazinebisacrylamide (which are particularly suitable for cross linking acylamides), phenylene diamine, dibromobutane, epichlorohydrin, trimethylolpropane trimethacrylate and N,N′-methylenebisacrylamide. The mole ratio of monomer to cross-linker is preferably from 1:1 to 1:15. See WO 02/00737 and WO 2006/120381 for further details of propofol receptors.

FIG. 2A shows the receptor layer as a MIP. The unfilled triangles represent the binding sites for the analyte. The binding sites are provided by synthesising the MIP in the presence of the analyte to be detected, or a close structural analogue of the analyte, using well-known techniques, rendering the MIP capable of selectively binding the analyte, see WO 2005/075995 and WO 2006/120381.

One of the main limitations associated with the development of sensors of this type has been the limited number of available binding sites in the receptor layer 4. This is particularly relevant where the receptor layer is a MIP. In a MIP for example, it is important to consider not just the total number of binding sites, but the number of binding sites able to communicate electrically from the transducer 6 via the receptor material to the analyte. In a MIP, there may be many binding sites, but due to the thickness of the MIP and its insulating nature, electronic communication with the analyte is prevented. The limited number of binding sites in electrical communication with the transducer 6 (via the receptor material) often reduces the sensitivity of the sensor and prohibits the detection of very low concentration of analytes. Another limitation lies in the electrically insulating nature of the receptor layer 4. Again this is particularly relevant for synthetic polymers, such as MIPs, and even more so when applied to electrochemical sensors. Tight control over the quantity of such material in the sensor is necessary in order to avoid the insulation of the sensor and loss of sensitivity.

The present invention addresses this problem by keeping the same material for the receptor layer 4, e.g. a MIP, but introducing a dispersed electrically conductive material. The conductive material is dispersed throughout the receptor material. It has been found that such a material is able to facilitate electronic communication between the analyte in a binding site and the bulk of receptor layer 4, and hence facilitate electronic communication between the analyte and the transducer 6 itself. Although not wishing to be bound by theory, it is thought that the entrapped conductive material is located in close proximity to the active binding sites which allows for better communication between the MIP, an insulator in nature, and the electrochemical transducer. Surprisingly, it seems able to do this without interfering significantly with the binding between the analyte and the receptor.

As shown in FIG. 2B, the receptor layer is rendered electrically conductive by the addition of a dispersed electrically conductive material 7 which provides enhanced sensitivity. The dispersed conductive material 7 is generally a solid. It is preferably a dispersed powder and is preferably selected from conductive carbon (such as carbon black or graphite), a metallic powder (e.g. gold, silver, copper, platinum etc), metallic nanoparticles (e.g. gold, silver, platinum), carbon-based nanoparticles (such as fullerenes, carbon nanotubes or spheres, or carbon powder) and/or conductive organic molecules. When carbon is used, the preferred particle size range depends on the properties of the carbon material. When carbon black is used, it is preferably a grade with high electrical conductivity, such as Cabot Corporation's Vulcan XC-72. This has particles under 10 μm, and a density of 1.7 to 1.9 g·cm⁻³. A loading of 0.1-5% w/w of carbon black in the prepolymer mixture may be suitable. Dispersion may be achieved by dispersing the conductive material in the pre-polymer solution prior to polymerisation. In this manner the conductive material becomes integrated in to the polymer matrix.

The conductive material may be surface-modified to facilitate its dispersion, e.g. by silylation. This may be particularly useful with carbon black particles. It may be carried out in a conventional manner, e.g.:
(a) forming a suspension of the carbon black in a non-reactive medium, such as xylene
(b) adding an excess of a silylating agent, such as HMSDS
(c) removing the reaction product of the silylating agent and the carbon black, and
(d) forming a dispersion of the reaction product in the desired solvent (in this case our prepolymer mixture).

We have also used gold particles, in particular monolayer-protected clusters of gold protected by alkanethiols as reported by Huang et al., (J. Electrochem. Soc., 150 (7)(2003) 412-417), and Hostetler et al., (Langmuir, 14 (1998), 17-30).

The Au particle size ranges from 1.5 to 5 nm and is controlled by the nature of the alkanethiols as well as the ratio of gold salt/alkanethiols. In general, in our present embodiment, gold nanoparticles precursors are firstly prepared and then mixed with the MIP precursor solution. The MIP is then synthesised prior to coalescence of the Au nanoparticles.

The novel receptor layer obtainable by dispersing the conductive material, preferably a powder, in the pre-polymer prior to polymerisation is a particularly preferred embodiment of the present invention. The receptor layer obtainable in this way particularly preferably incorporates a synthetic polymer or a MIP as the receptor material. Preferably the sample is treated with ultra-sound to aid dispersion. (Dispersion may be aided by use of one or more of sonication, a sonic homogenizator probe, and stirring.) Alternatively, the receptor material may be provided as a powder and the conductive material is dispersed through the powder.

The receptor layer 4 binds the analyte and the presence of the analyte is detected by the transducer 6. For the transducer 6 to be able to detect the electrical signal from the receptor layer 4, the receptor layer 4 must be in electronic communication with the transducer 6. The receptor layer 4 may be disposed directly on the transducer 6, or the receptor layer 4 may be proximal to the transducer 6 and electronic communication is established by the presence of an electrolyte or other electrically conductive material between the receptor layer 4 and the transducer 6.

The transducer 6 is itself preferably disposed on the substrate 5. The transducer 6 may be disposed on the surface of the substrate 5 or it may be disposed within the substrate 5. The transducer 6 and the receptor layer 4 may also constitute a single entity. For example, an electrode material may be screen-printed onto a suitable substrate 5. A polymer (forming the receptor material) and graphite (forming both the dispersed electrically conductive material 7 and the transducer 6) may then be combined and screen-printed onto the electrode material. The sensor 1 may also comprise further transducers and receptor layers to detect further analytes. The substrate 5 is preferably a planar substrate. The substrate 5 may be composed of silicon (e.g. a silicon wafer), ceramic, glass, metal, plastics etc. Alternatively, the receptor layer 4 itself may sufficiently resilient to act as a substrate and a separate substrate 5 is not required.

The transducer 6 may be any transducer which relies upon an electrical signal from the receptor layer 4. The transducer 6 is preferably an electrochemical transducer, and most preferably an amperometric transducer or a conductimetric transducer. Changes in current or resistance can be measured upon binding of the analyte to the sensor and related to the concentration of the analyte in the sample.

Preferably, the receptor layer has a sufficient capacity for the analyte to allow multiple or continuous use of the sensor.

In a particularly preferred embodiment of the present invention, the sensor 1 is used for the measurement of propofol in a blood sample, which employs a MIP as the receptor layer. More preferably, the MIP is immobilised on top of an amperometric transducer.

Preferably, the sensor 1 is used to oxidise the propofol being bound by the MIP. This can be achieved, for example, by operating the transducer as an amperometric transducer and applying a voltage of 0.35 V or larger between the working electrode and the reference electrode. By choosing this voltage carefully, i.e. just slightly above the level at which propofol can be oxidised, the oxidation of other species can be suppressed.

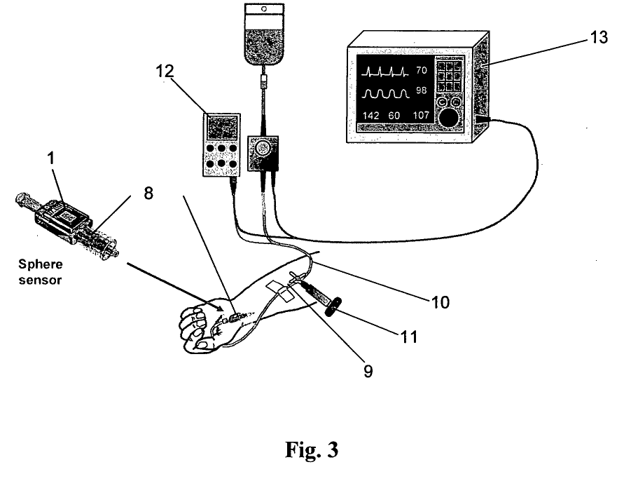

The sensor of the present invention is typically incorporated into a sampling system and a signal processing unit. Accordingly, the present invention also provides a sampling apparatus comprising a housing coupled to a sampling port and incorporating the sensor as described herein and a signal processing unit in electronic communication with the sensor. An example of such a system is shown in FIG. 3. The system is equipped with a housing 8 incorporating the sensor 1 coupled to a sampling port 9 in an intravascular line 10 above the sensor 1. A sampling device 11, for example, a syringe, is coupled to the sampling port 9. Using the sampling device 11, the user will withdraw blood flushing it across the sensor 1 in order to take a measurement. After the measurement is completed, the blood may be flushed back into the patient or it may be flushed to waste. In another embodiment, the sensor can be incorporated into the intravascular-flushing line, for example, along with one or more other sensors, such as a pressure sensor. Samples may be taken either periodically, regularly, event-driven, on demand or following a user intervention.

The sensor 1 is connected to a local display and signal processing unit 12 which may be connected to a patient monitoring device 13. The sensor 1 is also connected to the housing 8 electronically using techniques known in the art.

In addition to the system described above, the sensor may be employed in a range of other sensing systems, known to those skilled in the art. For example, rather than being directly connected to the patient, a sample may be taken from the patient and transported to and injected into an analyser, into which the sensor is integrated, for sample analysis.

The present invention also provides a method of detecting an analyte comprising providing a sample potentially containing the analyte, contacting the sample with the sensor as described herein, obtaining a signal, and processing the signal to provide an indication of the amount of the analyte present in the sample. The sample is preferably a fluid sample and most preferably a bodily fluid.

By providing detection and measurements of markers, substances or drugs, the sensor of the present invention provides feedback for the treatment of the patient based on the results of the analysis made. This feedback may be provided either directly to the user or it may be part of a closed-loop control system including the device administering the treatment to the patient. One particular example is a sensor for an anaesthetic agent, such as propofol, which measures the concentration of the anaesthetic agent in one or more bodily fluids or body compartments, e.g. blood or blood plasma, and based on these measurements directs, either directly or the user, the subsequent delivery of the anaesthetic agent, e.g. by controlling the rate of delivery to the patient via a syringe pump.

The sensor may also be used with systems which monitor other parameters which characterise the health of a patient, in particular markers indicating disease states or direct the patient's treatment, e.g. blood gases, pH, temperature etc.

### EXAMPLES

The present invention will now be described with reference to the following examples which are not intended to be limiting.

### Example 1

### Sensor Preparation

A sensor was prepared by microfabricating a sensor chip and depositing a MIP on the transducer using the methodology discussed in WO 2005/075995 and WO 2006/120381. Specifically, 50 mg of propofol, 210 mg of DEAEM (monomer), 1.3 g of ethylene glycol dimethacrylate (cross linker), and 31 mg of 2,2-dimethoxy-2-phenylacetophenone (free-radical polymerisation photoinitiator) were dissolved in 1.55 g of dimethylformamide. The pre-polymerisation mixture was further bubbled with nitrogen for 5 mins in order to remove any dissolved oxygen present in the mixture. The mixture was then added to 90 mg of Vulcan XC72R (conductive carbon black) (VULCAN is a trademark of Cabot Corporation) and sonicated for 5 mins with an ultrasound homogeniser in order to disperse the carbon particles. Approximately 40 nL of the pre-polymerisation mixture was then deposited onto a transducer comprising a platinum electrode and irradiated with UV radiation for 10 mins. The sensor was finally washed with 5 mL of 0.1 M HCl/20% methanol, rinsed with water, and washed with 5 mL of 0.1 M NaOH/20% methanol, rinsed with water, and finally blow dried in a stream of compressed air.

A further sensor was prepared using the same methodology but without the conductive carbon black particles (as represented in FIG. 2A).

### Example 2

### Sensor Evaluation

In order to assess the sensitivity enhancement produced by the introduction of conductive element in a propofol MIP, a bare amperometric sensor comprising a platinum electrode, a MIP-coated amperometric sensor and a conductive-MIP coated amperometric sensor, operated at a constant potential of +500 mV, were tested for their respective response to propofol in the concentration range 0-100 μM prepared in phosphate buffer saline pH 7.4. The results are summarised in Table 1.

TABLE 1
Propofol detection.

| | Sensor Response (nA/μM) |
|---|---|
| Bare sensor | 0.03 |
| MIP-coated sensor | 0.18 |
| Conductive MIP-coated sensor | 0.80 |

The MIP coating allowed for a six-fold increase in the sensitivity of the measurement of propofol when compared to a bare sensor. The MIP captures the propofol from the sample, concentrating propofol in the accessible binding sites of the MIP on the surface of the sensor electrode. The presence of the conductive carbon black in the MIP further improved the sensitivity of the sensor by a factor of 4.5, that is approximately 25 times more sensitive than a bare sensor.

1. A sensor for detecting an analyte in a sample comprising a transducer and a receptor layer in electrical communication with the transducer, wherein the receptor layer comprises a receptor material and a dispersed electrically conductive material.

2. A sensor as claimed in claim 1, wherein the sensor further comprises a substrate.

3. A sensor as claimed in claim 2, wherein the transducer is disposed on the substrate.

4. A sensor as claimed in claim 1, wherein the sensor further comprises a confinement structure, the confinement structure comprising a first limiting structure defining a first interior space, and wherein the transducer and the conductive molecularly imprinted polymer are disposed in the first interior space.

5. A sensor as claimed in claim 4, wherein the first limiting structure is a continuous structure.

6. A sensor as claimed in claim 4, wherein the first limiting structure is annular.

7. A sensor as claimed in claim 1, wherein the sensor further comprises a channel to contain the sample.

8. A sensor as claimed in claim 1, wherein the conductive material is a conductive powder.

9. A sensor as claimed in claim 1, wherein the receptor material is a synthetic polymer.

10. A sensor as claimed in claim 9, wherein the synthetic polymer is a molecularly imprinted polymer.

11. A sensor as claimed in claim 1, wherein the conductive material is selected from conductive carbon black, metallic power, metallic nanoparticles, carbon-based nanoparticles, conductive organic molecules and combinations thereof.

12. A sensor as claimed in claim 1, wherein the receptor layer has a sufficient capacity for the analyte to allow multiple or continuous use of the sensor.

13. A sensor as claimed in claim 1, wherein the transducer is an electrochemical transducer.

14. A sensor as claimed in claim 11, wherein the transducer is an amperometric or a conductimetric transducer.

15. A sampling apparatus comprising a housing coupled to a sampling port and incorporating the sensor as claimed in claim 1, and a signal processing unit in electronic communication with the sensor.

16. A method of detecting an analyte comprising providing a sample potentially containing the analyte, contacting the sample with the sensor as claimed in claim 1, obtaining a signal, and processing the signal to provide an indication of the amount of the analyte present in the sample.

17. A method as claimed in claim 16, wherein the sample is a fluid sample.

18. A method as claimed in claim 17, wherein the fluid sample is a bodily fluid.

19. A method of manufacturing a sensor according to claim 1 including a step of forming said receptor layer by providing a pre-polymer composition, dispersing said electrically conductive material in said pre-polymer composition, and effecting polymerisation of said pre-polymer composition to produce said receptor layer.
